(19) United States
(12) Patent Application Publication
(10) Pub. No.: US 2008/0033504 A1
(43) Pub. Date: Feb. 7, 2008

(54) METHOD OF RELIEVING MIGRAINES OR HEADACHES

(76) Inventor: Lawrence E. Bertolucci, Carlsbad, CA (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 11/763,305

(22) Filed: Jun. 14, 2007

Related U.S. Application Data
(63) Continuation of application No. 10/790,170, filed on Mar. 1, 2004, now abandoned.
Application No. 10/790,170 is a continuation of application No. 09/814,678, filed on Mar. 21, 2001, now abandoned.

(51) Int. Cl. A61N 1/00

(52) U.S. Cl. 607/46

(57) ABSTRACT

A device for providing noninvasive electrical stimulation of a single acupuncture site for treatment of migraines and/or headaches is disclosed.

### CROSS-REFERENCE TO RELATED APPLICATIONS

This application is a Continuation of U.S. NonProvisional patent application Ser. No. 10/790,170 filed Mar. 1, 2004, now pending; which claims the benefit of and is a Continuation of Ser. No. 09/814,678 filed Mar. 21, 2001, now abandoned.

### FIELD OF THE INVENTION

The methods and devices described below relate to the fields of treatment of migraines and/or headaches and noninvasive electrical stimulation of an acupuncture point.

### BACKGROUND OF THE INVENTION

A headache is pain that occurs in the tissues covering the brain, the attaching structures at the base of the brain, and the muscles and blood vessels around the scalp, face, and neck. The three most common headaches are tension, migraine, and cluster. Tension headaches are the most common and cluster headaches affect only about one-percent of the population, mostly males. The exact mechanism for each type of headache is not known. Some experts theorize that they all occur from the same mechanism.

Migraines are divided into two types, the common migraine and the classical migraine. The difference between the common and the classical migraine is whether or not the patient experiences the migraine aura prior to experiencing the headache. The migraine aura is a composite of possible symptoms, namely, visual disturbances, light sensitivity, speech difficulty, tingling of the face or hands, and confusion. The common migraine is not preceded by an aura, while the classical migraine is preceded by an aura.

Research scientists are unclear about the precise cause of migraine headaches. There seems to be a consensus, however, that the key element is blood flow changes in the brain. One theory states that the nervous system responds to a trigger such as stress by creating a spasm in the nerve-rich arteries at the base of the brain. The spasm close down or constricts several arteries supplying blood to the brain, including the scalp artery and the carotid arteries. As these arteries constrict, the flow of blood to the brain is reduced. At the same time, platelets clump together, this process is believed to cause the release of serotonin. Serotonin acts as a powerful constrictor of arteries, thus further reducing the blood supply to the brain. This reduction in blood flow is likely the cause of the migraine aura.

The reduced blood flow decreases the brain's supply of oxygen. Reacting to the reduced blood supply, certain other arteries within the brain dilate in an attempt to increase the blood supply and thus the oxygen levels in the brain. The dilation spreads and finally affects the carotid and scalp arteries. The dilation of these arteries triggers the release of pain-producing prostagladins. Prostagladins cause inflammation and swelling. Other substances which increase sensitivity to pain are also released. The circulation of these chemicals and the dilation of the scalp arteries stimulate the pain-sensitive nociceptors. The result, a throbbing headache.

Acupuncture has long been used in the treatment of migraines and/or headaches. In accordance with well-known acupuncture standards, several acupuncture points are simultaneously stimulated to achieve the therapeutic goal. As taught in The Basics of Acupuncture by Stux and Pomeranz, Springer-Verlag, New York, pp. 237-238, 1995, the specific acupuncture points being used to treat the migraine or headache depends upon the where the patient is experiencing pain.

If the pain is along the gallbladder channel, then ten acupuncture points are stimulated: the top of the head (Du 20 Baihui), the forehead (GB.14 Yangbai), behind the ear (GB.20 Fengchi), above the ear (GB.8 Shuaigu), dorsal-side of lower arm (SJ.5 Waiguan), top of the hand between the thumb and index finger (LI.4 Hegu) , the toe (GB.41 Linqi), the ankle (GB.37 Guangming), top of the foot (St.44 Neiting), and the foot (Liv.3 Taichong). If the pain is in the area of the temple, then the top of the head (Du 20 Baihui), near the top of the head (St.8 Touwei), the temple (GB.4 Hanyan), top of the hand between the thumb and index finger (LI.4 Hegu)˜the elbow (LI.11 Quchi), top of the foot (St.44 Neiting), and the shin (St.36 Zusanli) are stimulated. If the pain is along the urinary bladder channel, then the top of the head (Du 20 Baihui), eyebrow (UB.2 Zanzhu), back of the neck (UB.10 Tianshu), the side of the hand near the pinkie finger (SI.3 Houxi), top of the hand between the thumb and index finger (LI.4 Hegu), the ankle (UB.60 Kunlun), and the little toe (UB.67 Zhiyin) are stimulated. If the pain is in the area of vertex Du 20 Baihui, then the top of the head (Du 20 Baihui), top of the head (Ex.6 Sishencong), the abdomen (Liv.14 Qimen), top of hand between the thumb and index finger (LI.4 Hegu) , dorsal-side of lower arm (SJ.6 Zhigou), the foot (Liv.3 Taichong), top of the foot (Liv.2 Xingjian), and the calf (GB.34 Yanlingquan) are stimulated. All the acupuncture points listed for each area of pain are stimulated simultaneously to obtain results.

Bertolucci, Nausea Control Device, U.S. Pat. No. 4,981,146, Jan. 1, 1991, describes a nausea control device in the form of a watch-like housing attachable to the human wrist by an adjustable attachment band. The device uses non-invasive nerve stimulation whereby electricity is passed through two electrodes to stimulate nerves located on the ventral side of the wrist (this anatomical position is sometimes referred to as the palmar side of the wrist). The treatment provided by the device is sometimes referred to as electro-acupuncture which is a form of acupuncture, and the ventral site of application is referred to in the acupuncture art as the P6 point, pericardium 6 point, or master point of the pericardium meridian (sometimes referred to as the vascular meridian). A primary object of the invention is to provide a non-chemical, non-invasive, painless and inexpensive method of alleviating nausea. It is also portable, self-contained and convenient to the patient. Electrical pulse repetition rate of approximately 70 pulses per second, and a pulse width of 80 microseconds has been found to provide effective relief of nausea in a patient. Our currently preferred electrical pulse pattern comprises about 350 microsecond pulse width at about 31 pulses per second at power levels of about 10-35 milli-amps peak pulse height. Thus a wide range of pulse patterns may be used in noninvasive nerve stimulation devices.

We have discovered that using noninvasive electrical stimulation of the P6 or Neiguan point of the pericardium meridian relieves migraines and/or headaches.

### SUMMARY OF INVENTION

The method described below employs use of the device described in Bertolucci, Nausea Control Device, U.S. Pat. No. 4,981,146 (Jan. 1, 1991), and similar devices, for the relief and alleviation of migraines and/or headaches.

### BRIEF DESCRIPTION OF THE DRAWINGS

FIG. 1 illustrates placement of an electro-acupuncture device over the P6 acupuncture point on the human wrist.

FIG. 2 illustrates a stimulation waveform for stimulating the wrist in accomplishing the treatment.

FIG. 3 illustrates an individual pulse of the stimulation waveform.

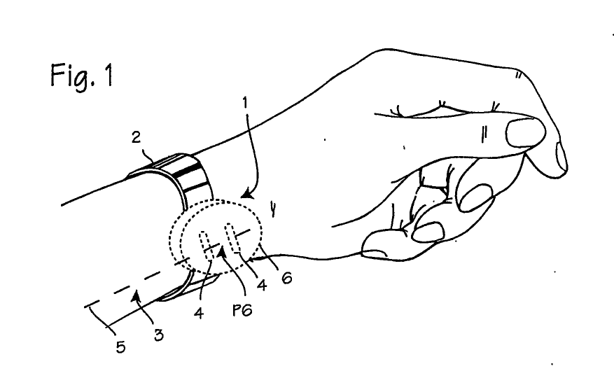

### DETAILED DESCRIPTION OF THE PREFERRED EMBODIMENT

Use of our ReliefBand® NST™ device for the approved treatment of nausea has revealed that the treatment also relieves migraines and/or headaches. Significant reduction in migraines and/or headaches has been observed when electrostimulation is provided to the P6 point on the wrist. The ReliefBand® NST™ is a wristwatch like device worn on the wrist and energized to provide electrical stimulation to the wrists. The ReliefBand® NST™ non-invasive nerve stimulation device 1 is secured with strap 2 to the ventral side of the wrist 3 such that the pair of electrodes 4 are disposed over the median nerve 5 (indicated by the phantom line) in contact with the skin in the vicinity of the p6 acupuncture point. The electrodes are on the underside of the housing 6, the required battery and control electronics are housed within the housing, and input mechanisms are located on the outer face of the housing. The electrodes stimulate the median nerve and collateral or associated nerve structures.

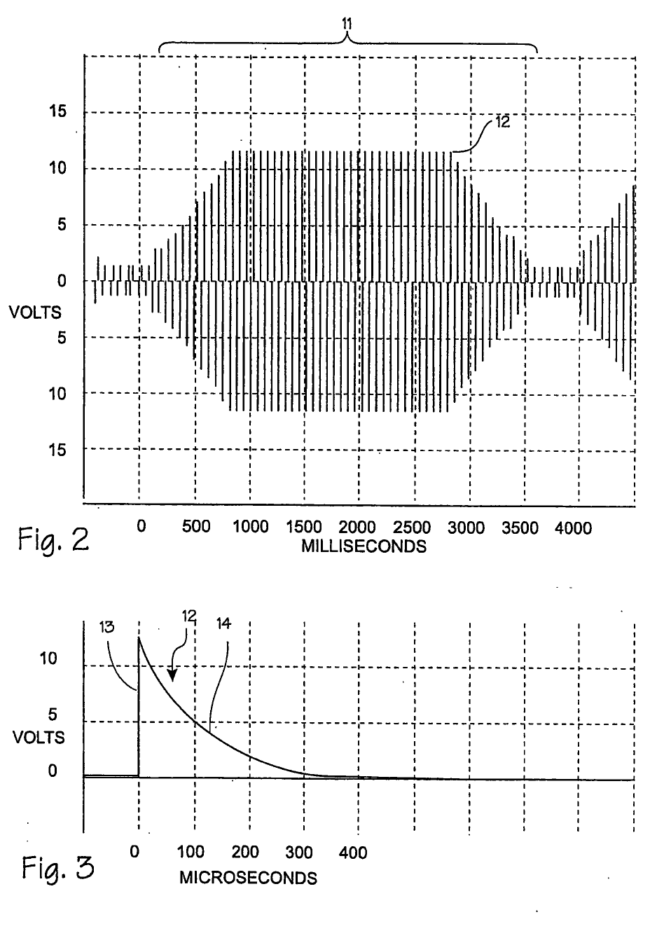

FIG. 2 shows the preferred waveform. The overall waveform comprises a series of bipolar trapezoidal waveforms which make low frequency pulses 11. The waveform is initiated at low power levels of about 1 to 2 volts and ramps up over a period of about 1 second to a maximum level of 10-20 volts, and is maintained for about 2 seconds, and then ramps down over a period of about 1 second to low power levels of about 1 to 2 volts. The individual pulses 12 are separated by about 32 milliseconds (msec) (measured peak to peak), and last about 350 microseconds (μsec). The individual pulses alternate between negative and positive pulses, and are said to constitute a bipolar waveform. The individual pulses are illustrated in FIG. 3, in which the time scale is enlarged to show the detail. The individual pulse 12 is made of a sharply vertical spike which decays exponentially over a period of about 350 μsec, thus comprising a basically vertical leading edge 13 and an exponentially decaying trailing edge 14 to each individual pulse. The following pulse will be shaped the same, except that it will be of negative voltage. The exponential nature of the individual pulse decay maximizes the high frequency components in the signal. These high frequency components contribute to a lessening of the skin impedance, in particular the capacitive components. This contributes to a higher level of current able to enter the deeper tissues. The power levels may be adjusted up or down to intensify the therapeutic effect of the device or lessen the sensation causes by the device, according to the preferences of individual users. The pulse rate within the waveform may be increased or decreased also.

To use the device to alleviate migraines and/or headaches, the user merely secures the housing over the inner surface of the wrist and straps it on like a wristwatch. This places the electrodes over the P6 acupuncture point, in electrical contact with the skin overlying the median nerve. The user then turns the device on, adjusts it to a comfortable power level, and allows stimulation to continue for a few minutes, for example 5-10 minutes to achieve relief. The device may be applied intermittently, once every hour or so, or continuously. The device provides electrical current and voltage to the electrodes which stimulates the P6 acupuncture point. While less convenient, the methods may be accomplished with electro-acupuncture needles or electrodes handled individually by an acupuncturist.

While the devices and methods have been described in reference to the environment in which they were developed, they are merely illustrative of the principles of the inventions. Other embodiments and configurations may be devised without departing from the spirit of the inventions and the scope of the appended claims.

In the Claims:

1. A method of relieving a headache in a patient comprising the steps of:
mounting a non-invasive nerve stimulation device onto the ventral side of the wrist;
generating a stimulation signal; and
delivering the stimulation signal to the ventral side of the wrist.

2. A method of relieving a headache in a patient comprising the steps of:
mounting at least one electrode onto the ventral side of the wrist;
generating a stimulation signal; and
delivering the stimulation signal to said at least one electrode to stimulate the ventral side of the wrist.

3. The method of claim 2 wherein said mounting step includes providing a wristwatch-like housing carrying the electrodes, and providing securing means for mounting the housing onto the wrist, with the housing having a circuit means for generating the stimulation signal encased within the housing.

4. The method of claim 2 wherein said delivering step comprises delivering an intermittent stimulation signal.

5. The method of claim 2 wherein said delivering step comprises delivering a continuous stimulation signal.

6. A method of relieving a headache in a patient by stimulating the median nerve and associated nerve structures in the wrist of the patient with electrical energy.

7. A method of relieving a headache in a patient comprising the steps of:
applying an electro-acupuncture device to the wrist; and
stimulating the wrist with the electro-acupuncture device.

8. A method of relieving a migraine In a patient comprising the steps of:
mounting a non-invasive nerve stimulation device onto the ventral side of the wrist;
generating a stimulation signal; and
delivering the stimulation signal to the ventral side of the wrist.

9. A method of relieving a migraine in a patient comprising the steps of:
mounting at least one electrode onto the ventral side of the wrist;
generating a stimulation signal, and
delivering the stimulation signal to said at least one electrode to stimulate the ventral side of the wrist.

10. The method of claim 9 wherein said mounting step includes providing a wristwatch-like housing carrying the electrodes, and providing securing means for mounting the housing onto the wrist, with the housing having a circuit means for generating the stimulation signal encased within the housing.

11. The method of claim 9 wherein said delivering step comprises delivering an intermittent stimulation signal.

12. The method of claim 9 wherein said delivering step comprises delivering a continuous stimulation signal.

13. A method of relieving a migraine in a patient by stimulating the median nerve and associated nerve structures in the wrist of the patient with electrical energy.

14. A method of relieving a migraine in a patient comprising the steps of:
applying an electro-acupuncture device to the wrist; and
stimulating the wrist with the electro-acupuncture device.
